(19) United States
(12) Patent Application Publication
(10) Pub. No.: US 2006/0036168 A1
(43) Pub. Date: Feb. 16, 2006

(54) Electrohydraulic shock wave-generating system with automatic gap adjustment

(76) Inventors: Shen-Min Liang, Tainan (TW); Yong-Ren Pu, Kwei-Jen (TW); Ioannis Manousakas, Kaohsiung City (TW); Chia Hui Wang, Dou-Liu City (TW); Tzu-Liang Chen, Tainan (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/896,040

(22) Filed: Jul. 22, 2004

(51) Int. Cl. A61B 8/00

(52) U.S. Cl. 600/437

(57) ABSTRACT

An electrohydraulic shock wave-generating system for extracorporeal therapy of renal stones or musculoskeletal disorders includes a shock wave generator, a micro high-sensitivity camera, and a gap-controlling unit. The shock wave generator includes a truncated ellipsoidal bowl and two electrodes, each electrode having a portion inside the bowl, with a gap being defined between the electrodes. The micro high-sensitivity camera acquires an image of the electrodes for finding a size of the gap. The gap-controlling unit controls the size of the gap and moves at least one of the electrodes to adjust the size of the gap. A medical treatment for fragmenting stones or for curing musculoskeletal disorders can be carried out without increasing the operational voltage applied to the electrodes under gap control provided by the system. The system also includes a computer control unit to provide automatic control of the gap.

### BACKGROUND OF THE INVENTION

1. Field of the Invention

The present invention relates to an electrohydraulic shock wave-generating system for extracorporeal therapy of renal stones or musculoskeletal disorders. In particular, the present invention relates to a system that generates underwater shock waves focused on a target in a patient for effective treatment of the target.

2. Description of the Related Art

In the last two decades extracorporeal shock wave lithotripsy (ESWL) provides a new way to treat renal stones in the human body; namely, the traditional invasive surgery for removing the stone out of the patient's body has been replaced by noninvasive extracorporeal lithotripsy using an apparatus that generates shock waves to break the renal stone into smaller pieces. Although the extracorporeal therapy for renal stones has been widely used and accepted by the patients, the successful stone-fragmenting rate under human monitoring was found at best 60-70%, as the stone in the body of the patient receiving the therapy moved when the patient breathed such that the shock waves could not precisely hit the stone. To solve this problem, U.S. patent application Ser. No. 10/061,240 proposes a system for tracing a renal stone during medical treatment.

Extracorporeal shock wave lithotripters currently available on the market include electrohydraulic type, electromagnetic type, and piezoelectric type. The electrohydraulic lithotripters are more widely used than the other two types, as electrohydraulic lithotripters had been invented earlier. Electrohydraulic lithotripters generate shock waves with high-energy flux density (intensity), while the electromagnetic- and piezoelectric-type generate low-energy flux density. During a treating process, about 3000 shock waves are generated for an electrohydraulic lithotripter and about 5000-6000 shock waves for an electromagnetic lithotripter or a piezoelectric lithotripter, both of which take a relatively long time for producing the desired number of shock waves. As a result, most doctors and patients dislike the long treatment process for stone therapy.

For most of electrohydraulic lithotripters, the gap between two electrodes of the shock wave generator is fixed and thus could not be adjusted. Only few of them allow manual adjustment. Problems occur in a case that the gap between the electrodes is not controlled in a predetermined range. More specifically, the more times the shock waves are fired, the larger the gap between the electrodes is. Thus, when the gap between the electrodes is larger than a threshold, the intensity of the shock waves becomes weak. Even worse, no shock waves can be fired. A remedy to this problem is to increase the voltage to an extent sufficient to generate shock waves or to replace the electrodes by new ones, yet the shock wave intensity and the times for fragmenting the stones cannot be controlled, leading to a low stone-fragmenting efficiency.

However, even the gap between the electrodes is manually adjusted, no instant and effective monitoring/measuring device is provided. Hence, the patient has to move away from the shock wave reflector (a truncated ellipsoidal bowl) to allow measurement of the gap between the electrodes by X-ray or other methods. The treating process is interrupted and brings inconvenience to the patient receiving therapy.

In addition to the application of ESWL to the urology, extracorporeal shock wave therapy (or orthotripter) for musculoskeletal disorders, such as calcific tendonitis of shoulder, tennis elbows, epicondylitis, plantar fasciitis, delayed unions, and nonunion fractures, has also been used in recent years. Since electrohydraulic orthotripters use the same principle of underwater shock wave focusing as lithotripters, the present invention can be applied to the orthotripters.

### SUMMARY OF THE INVENTION

An objective of the present invention is to provide an electrohydraulic shock wave-generating system for extracorporeal therapy of renal stones or musculoskeletal disorders that, given the same operational voltage, generates underwater shock waves with uniform intensity, reducing the number of the shock waves needed for treating a target and thus the time for the target treatment, and reducing injury to the tissue of the patient. This objective is achieved by means of automatically adjusting the gap between two electrodes of a shock wave generator of the system. The adjustment of the gap between the electrodes can be done, regardless of the material of the electrodes used. The softer the material of the electrodes is, the more frequently the gap between the electrodes is adjusted. Thus, given different operational voltage, the times of firing the shock waves for treating the stones can be effectively controlled and the stone-fragmenting efficiency is improved.

Another objective of the present invention is to provide a system that can be incorporated with currently used extracoporeal shock wave therapy machines such as extracorporeal shock wave lithotripters for treating renal calculi and extracorporeal shock wave orthotripters for treating musculoskeletal disorders.

In accordance with an aspect of the invention, an electrohydraulic shock wave-generating system for extracorporeal therapy of renal stones or musculoskeletal disorders comprises a shock wave generator, a micro high-sensitivity camera, and a gap-controlling unit. The shock wave generator includes a bowl (a shock wave reflector) and two electrodes, each electrode having a portion inside the bowl, with a gap being defined between the electrodes. The micro high-sensitivity camera acquires an image of the electrodes for finding the size of the gap. The gap-controlling unit controls the size of the gap and moves at least one of the electrodes to adjust the size of the gap. It was found that a truncated ellipsoidal bowl with eccentricity of approximately 0.71 produces a best result of focusing pressure at a geometric focus where a target (e.g., a kidney stone) is located.

In an embodiment of the invention, the shock wave generator includes a base on which the bowl is mounted. The base includes a transparent window through which the image of the electrodes is acquired by the micro high-sensitivity camera.

The gap-controlling unit includes two servomotors and two servomotor drivers for driving the servomotors. In an embodiment of the invention, the gap-controlling unit includes a multi-axis control card to thereby control the gap between the electrodes. The gap-controlling unit includes two transmission assemblies each including a first member driven by an associated servomotor and a rotatably supported second member. Each electrode is coupled to the second member of an associated transmission assembly such that rotation of each servomotor causes rectilinear movement of the electrode along a longitudinal direction of the electrode. In an embodiment of the invention, the first member and the second member are pulleys with an endless belt mounted around the pulleys. Each electrode is fixed to a copper base that is connected to one of the pulleys and rotatably supported by a fixed seat.

The system includes a computer control unit programmed to compare the size of the gap between the electrodes with an optimal gap size and to activate the gap-controlling unit when a difference between the size of gap of the electrodes and the optimal gap size is greater than a threshold. The computer control unit may include a program based on C language or other computer graphic languages.

Moreover, the system includes an image-grabbing card with which the micro high-sensitivity camera is coupled. The system also includes an I/O card for controlling the voltage setting and shock wave firing of a shock wave generator.

Other objectives, advantages, and novel features of the invention will become more apparent from the following detailed description when taken in conjunction with the accompanying drawings.

### BRIEF DESCRIPTION OF THE DRAWINGS

FIG. 1 is a schematic view of an electrohydraulic shock wave-generating system for extracorporeal therapy of renal stones or musculoskeletal disorders in accordance with the present invention.

FIG. 2 is a perspective view of a shock wave generator of the system in accordance with the present invention.

FIG. 3 is a top view of the shock wave generator in FIG. 2.

FIG. 4 is a side view of the shock wave generator in FIG. 2.

FIG. 5 is a flowchart illustrating a process for gap control by means of feedback signals.

FIG. 6 is a flowchart illustrating a medical treatment process using the system in accordance with the present invention.

FIG. 7A is a diagram showing the curve of focusing pressure with gap control between two electrodes of the shock wave generator.

FIG. 7B is a diagram showing the curve of focusing pressure without gap control between the electrodes of the shock wave generator.

### DETAILED DESCRIPTION OF THE PREFERRED EMBODIMENT

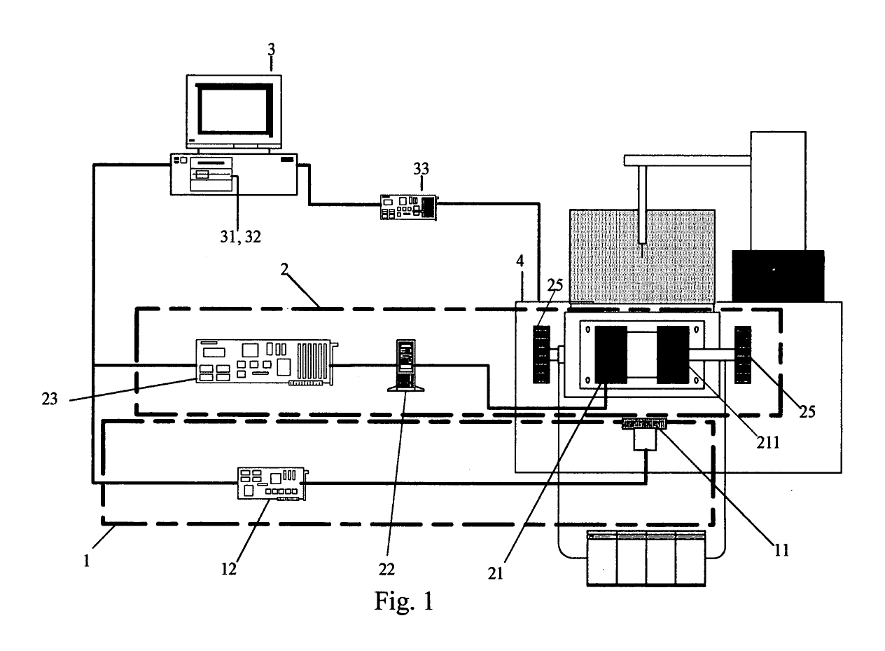

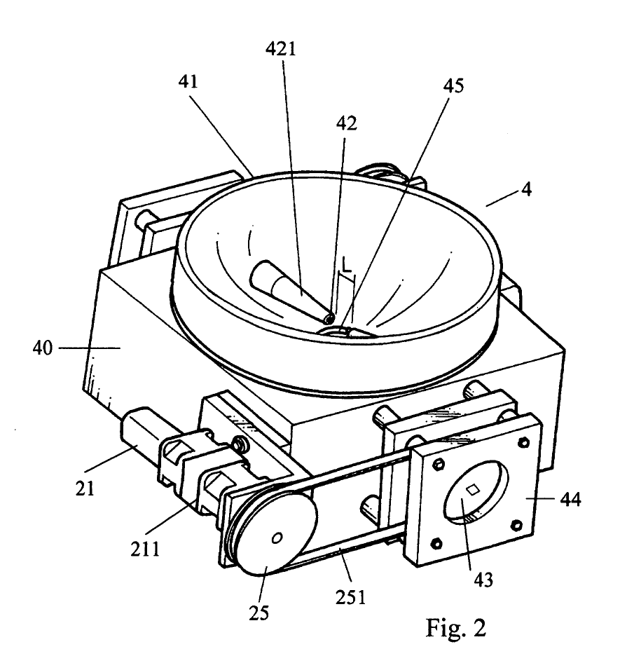

Referring to FIG. 1, an electrohydraulic shock wave-generating system for extracorporeal therapy of renal stones or musculoskeletal disorders in accordance with the present invention comprises a gap image grabbing/feeding unit 1, a gap-controlling unit 2, a computer control unit 3, and a shock wave generator 4 (see FIG. 2).

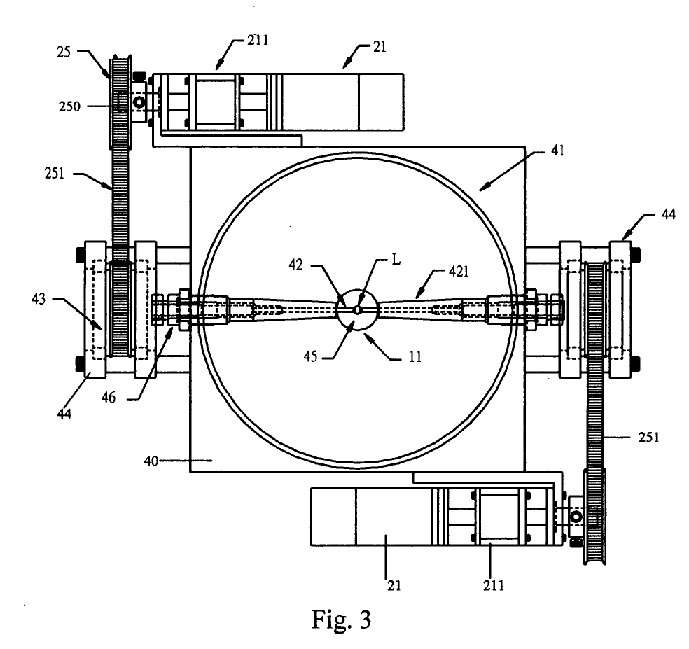

The gap image grabbing/feeding unit 1 includes a micro high-sensitivity camera 11 and an image-grabbing card 12. The camera 11 is mounted below a base 40 (see FIG. 3) on which a truncated ellipsoidal bowl 41 of the shock wave generator 4 is mounted, as shown in FIG. 3. The base 40 includes a transparent window 45 to allow measurement of the gap size between two electrodes 42 after erosion as a result of firing shock waves. The detected gap image is analyzed and processed by a program 32 for analyzing the gap size between the electrodes 42. A feedback signal is sent after processing by the program 32 to drive the gap-controlling unit 2.

Referring to FIGS. 1 through 3, the gap-controlling unit 2 includes at least one servomotor 21 (two in this embodiment), at least one reducer 211 (two in this embodiment), at least one servomotor driver 22 (two in this embodiment) for driving the servomotor 21, and at least one transmission assembly 25 (two in this embodiment). In this embodiment, each servomotor driver 22 is connected to a multi-axis control card 23. The multi-axis control card 23 is an interface card having a built-in motor microprocessor for controlling the servomotors 21, thereby controlling the gap between two electrodes 42. Each transmission assembly 25 includes a first pulley 250 connected to and driven by an associated reducer 211, which, in turn, is connected to and driven by an associated servomotor 21. Each transmission assembly 25 includes a second pulley 43, with an endless belt 251 mounted around the first pulley 250 and the second pulley 43, which will be described later.

The computer control unit 3 includes a program 31 for controlling the gap between the electrodes 42 and the program 32 for analyzing the gap size between the electrodes 42 mentioned above. The programs 31 and 32 are written into the memory of the computer control unit 3. The computer control unit 3 is also connected to the shock wave generator 4 through the I/O card 33 for controlling the voltage setting and shock wave firing of the shock wave generator 4.

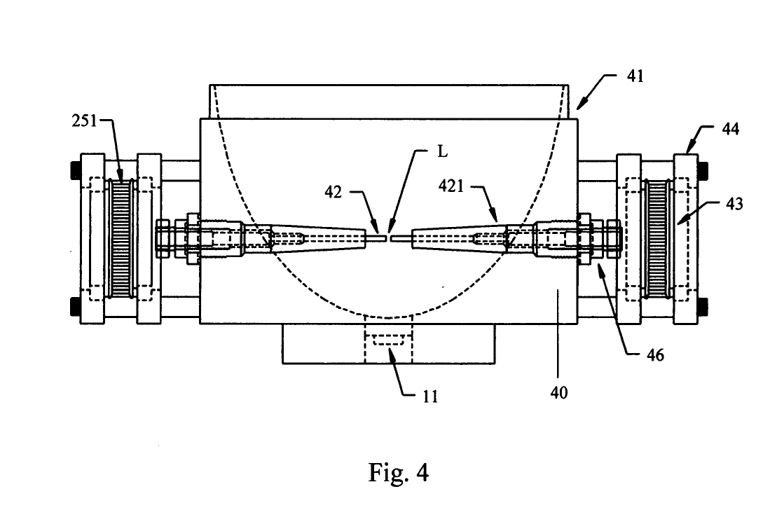

Referring to FIGS. 2 through 4, the shock wave generator 4 includes the bowl 41 and the electrodes 42, as mentioned above. In order to avoid electricity leakage in water, a portion of each electrode 42 inside the bowl 41 wears a jacket 421 made of Teflon. The pulley 43 of each transmission assembly 25 is connected to a copper base 46, which, in turn, is rotatably supported by a fixed seat 44 made of Bakelite. Each electrode 42 is welded to an associated copper base 46. When each servomotor 21 turns, the associated copper base 46 and the associated electrode 42 are turned and moved along a longitudinal axis of the electrode 42, thereby adjusting the distance, L, (i.e., the gap) between the electrodes 42.

The pulleys 250 and 43 and the endless belt 251 can be replaced with any other suitable members, such as gears and chain. Furthermore, other transmission assemblies can be used without departing from the scope of the invention. The reducers 211 can be omitted whenever appropriate. The servomotors 21 can be replaced with other suitable means for moving the electrodes 42 toward or away from each other. For example, the gap between the electrodes 42 can be adjusted through hydraulic or pneumatic control. Moreover, it is noted that the gap between the electrodes 42 can be adjusted through use of a set of a servomotor 21, a reducer 211, a servomotor driver 22, and a transmission assembly 25. In this case, the middle of the gap can be adjusted to a focus of the bowl 41 for subsequent firing of shock waves.

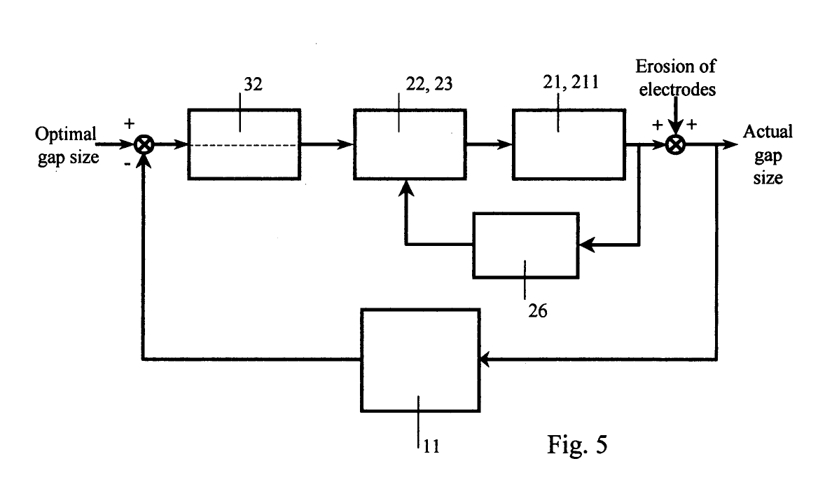

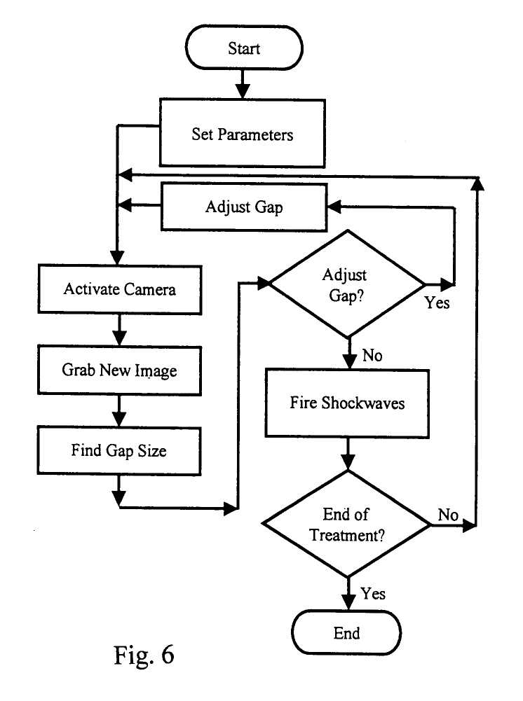

The computer control unit 3 uses programs to control the gap between the electrodes 42 (see FIG. 5) and to control medical treatments (see FIG. 6). The programs used by the computer control unit 3 may include a C language based program. Referring to FIG. 5, an optimal gap size based on experimental results is set, and the actual gap size is determined through the electrode image detected by the camera 11. Information relating to the actual gap size is sent to the computer control unit 3 and analyzed by the program 32. An encoder 26 is provided for transmitting the data of displacement, rotation speed, and so on, of the servomotors 21 with reducers 211 to a servo-controller (including the above-mentioned servomotor driver 22 and multi-axis control card 23). If the difference between the actual gap size and the optimal gap size exceeds a threshold, the result of comparison is sent to the servo-controller to activate the servomotors 21, moving the electrodes 42 toward each other until the difference between the actual gap size and the optimal gap size is not greater than the threshold. The camera 11 acquires the image of the electrodes 42 and thus determines the actual gap size between the electrodes 42 after erosion of the electrodes 42 as a result of firing shock waves. The procedure continues to keep the actual gap size in an acceptable range, allowing firing of shock waves without increasing the voltage applied to the electrodes 42.

FIG. 6 shows a flowchart illustrating a medical treatment process using the system for extracorporeal shock wave lithotripsy in accordance with the present invention. Firstly, parameters are prescribed, which include a summing parameter for counting the times of firing of shock waves (calculated by the computer control unit 3), the total number of shock waves required for the patient decided by the doctor in charge of the medical treatment process, an optimal gap size between the electrodes obtained from experiments, the threshold of difference between the actual gap size and the optimal gap size, etc.

Secondly, the camera 11 is activated to acquire the image of the electrodes 42 to find the actual gap size. The actual gap size is compared with the optimal gap size. If the difference between the actual gap size and the optimal gap size is smaller than the threshold, shock wave is fired. If the difference between the actual gap size and the optimal gap size is greater than the threshold, no shock wave is fired and the distance between the electrodes 42 is adjusted. The procedure continues until the treatment ends, i.e., the summing parameter (the number of the fired shock waves) equals the total number of shock waves required for the patient decided by the doctor. The medical treatment process is thus carried out without increasing the voltage applied to the electrodes 42.

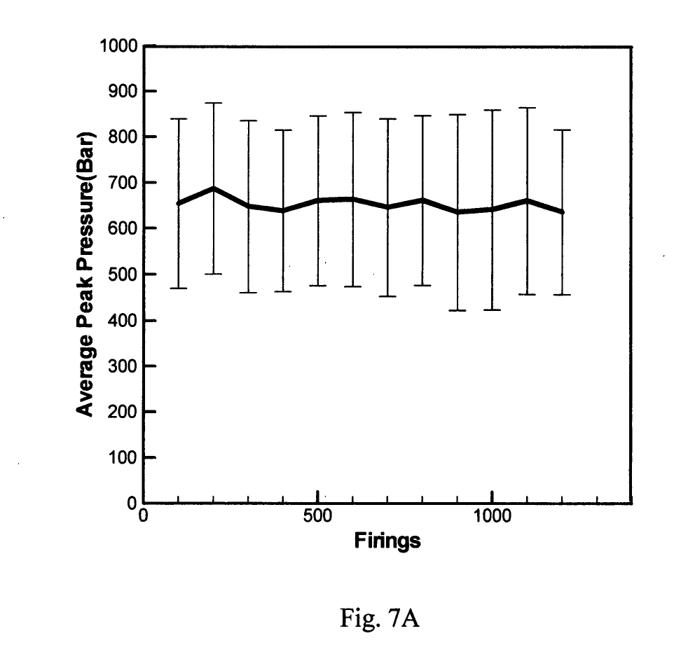

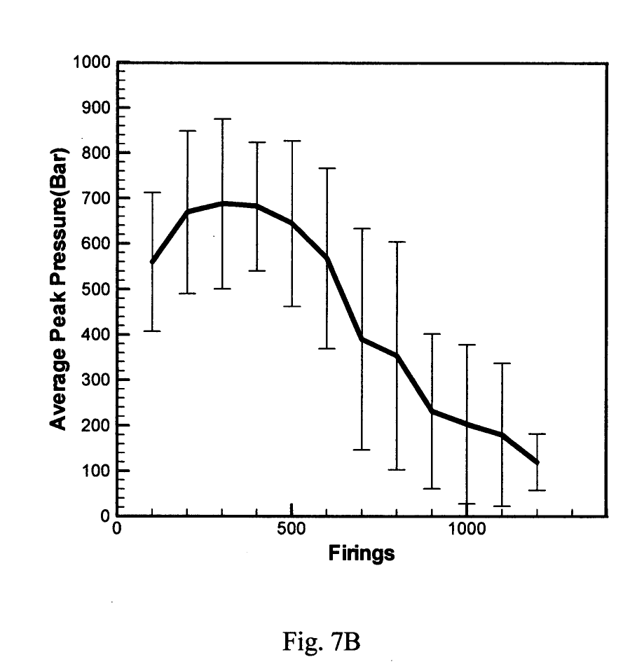

FIG. 7A is a diagram showing the curve of focusing pressure with gap control between the electrodes 42 of the shock wave generator 4. FIG. 7B is a diagram showing the curve of focusing pressure without gap control between the electrodes 42 of the shock wave generator 4. The electrodes 42 are made of bronze. As readily apparent from FIGS. 7A and 7B, stable pressure output can be obtained with gap control. The average peak pressure after firing 500 shock waves under gap control is far greater than that without gap control. It was found that for the bronze electrodes and at a voltage setting of 8 kV, after firing 1500 shock waves, the lithotripsy (or stone-fragmenting) efficiency with gap control is 55%, which is almost twice of that without gap control. The lithotripsy efficiency is defined as the overall weight of fragments smaller than 2 mm divided by the overall weight of the stones before the medical treatment.

The electrode 42 used in the present invention may have a length tenfold of that of a conventional one. Thus, a pair of electrodes 42 may be used to treat ten patients, while a pair of conventional electrodes can be used to treat only one patient.

In addition, the medical treatment can be carried out without increasing the operational voltage applied to the electrodes 42. Given the same operational voltage, the system in accordance with the present invention generates shock waves with uniform intensity, reducing the times for generating the shock waves and reducing injury to the tissue of the patient, as the gap between the electrodes 42 can be automatically adjusted. Adjustment of the gap between the electrodes 42 can be done regardless of the material of the electrodes used. The softer the material of the electrodes is, the more frequently the gap between the electrodes is adjusted. Thus, given different operational voltage, the number of shock waves for treating the stone can be effectively controlled and the stone-fragmenting efficiency is improved.

Moreover, the system in accordance with the present invention can be used in any extracoporeal shock wave lithotripters or orthotripters. Orthotripters aim at the treatments of musculoskeletal disorders such as calcific tendonitis of shoulder, tennis elbows, epicondylitis, plantar fasciitis, delayed unions, and nonunion fractures.

Although the invention has been explained in relation to its preferred embodiment, it is to be understood that many other possible modifications and variations can be made without departing from the scope of the invention as hereinafter claimed.

What is claimed is:

1. A system for generating underwater shock waves, comprising:
a shock wave generator including a truncated ellipsoidal bowl and two electrodes, each said electrode having a portion inside the bowl, with a gap being defined between the electrodes, and with a middle of the gap being located in a focus of the bowl;
a micro high-sensitivity camera for acquiring an image of the electrodes for finding a size of the gap; and
a gap-controlling unit for controlling the size of the gap, the gap-controlling unit including means for moving at least one of the electrodes to adjust the size of the gap.

2. The system as claimed in claim 1, with the shock wave generator including a base on which the bowl is mounted, the base including a transparent window through which the image of the electrodes is acquired by the micro high-sensitivity camera.

3. The system as claimed in claim 1, with the gap-controlling unit including two servomotors and two servomotor drivers for respectively driving the servomotors.

4. The system as claimed in claim 3, with the gap-controlling unit including two transmission assemblies, each said transmission assembly including a first member driven by an associated one of the servomotors and a rotatably supported second member, each said electrode being coupled to the second member of an associated one of the transmission assemblies such that rotation of each said servomotor causes rectilinear movement of the electrode along a longitudinal direction of the electrode.

5. The system as claimed in claim 1, with the system including a computer control unit programmed to compare the size of the gap between the electrodes with an optimal gap size and to activate the gap-controlling unit when a difference between the size of gap of the electrodes and the optimal gap size is greater than a threshold.

6. The system as claimed in claim 5, with the computer control unit including a C language based program.

7. The system as claimed in claim 3, with the gap-controlling unit including a multi-axis control card for controlling the servomotors to thereby control the gap between the electrodes.

8. The system as claimed in claim 1, with the system including an image-grabbing card with which the micro high-sensitivity camera is coupled.

9. The system as claimed in claim 4, with the first member being a first pulley, with the second member being a second pulley, with an endless belt mounted around the first pulley and the second pulley, with each said electrode being fixed to a copper base, with the copper base being connected to the second pulley and rotatably supported by a fixed seat.

10. The system as claimed in claim 1, with the truncated ellipsoidal bowl with eccentricity of approximately 0.71.
